(19) United States
(12) Patent Application Publication
(10) Pub. No.: US 2010/0274105 A1
(43) Pub. Date: Oct. 28, 2010

(54) Body Cavity Physiological Measurement Device

(76) Inventor: Beth Rosenshein, Superior, CO (US)

Correspondence Address: MARSH, FISCHMANN & BREYFOGLE LLP, 8055 East Tufts Avenue, Suite 450, Denver, CO, 80237, US

(21) Appl. No.: 12/766,598

(22) Filed: Apr. 23, 2010

Related U.S. Application Data
(60) Provisional application No. 61/172,046, filed on Apr. 23, 2009.

(51) Int. Cl. A61B 5/00

(52) U.S. Cl. 600/301

(57) ABSTRACT

Provided herein is a self-contained physiological measuring device adapted for disposition within a patient body cavity, primarily the vagina, for an extended period of time (e.g., 6-48 hours or more). While disposed within the body cavity, the device periodically measures one or more physiological parameters. In addition to measuring such physiological parameters, the device is operative to store such measurements to memory for subsequent download/processing upon removal of the device from the body cavity and/or upon wireless interrogation.

### CROSS-REFERENCE TO RELATED APPLICATION

This application claims priority and the benefit of the filing date under 35 U.S.C. 119 to U.S. Provisional Application No. 61/172,046, entitled, “BODY CAVITY PHYSIOLOGICAL MEASUREMENT DEVICE,” filed on Apr. 23, 2009, the contents of which are incorporated herein as if set forth in full.

### FIELD

The presented inventions are directed toward a method and system for monitoring one or more physiological parameters over an extended period of time. One aspect is directed towards a self-contained physiological monitoring device for disposition within a body cavity to make physiological measurements over an extended period of time. A further aspect is directed to the identification and treatment of hypogonadism.

### BACKGROUND

One medical condition that has heretofore resisted objective medical diagnosis, at least in the case of women, is hypogonadism. Hypogonadism is when the sex glands produce little or no hormones. In men, these glands (gonads) are the testes; in women, they are the ovaries. Diagnosis of physiologic changes associated with hypogonadism and profound hypogonadism in women and men, particularly in women, is generally limited to verbal inquiries of changes to the genitalia and sexual function. Without effective diagnostic screening tools a diagnosis of hypogonadism may be missed. Blood tests are generally not used to diagnose women because eugonadal and hypogonadal reference levels are not delineated, leading many physicians to not be able to diagnose hypogonadism.

It is desirable to be able to diagnose hypogonadism because, whether mild or profound, this illness creates significant changes to every major organ system in the body. Left untreated, over time hypogonadism typically has significant negative effects on cognitive function, endothelial function, neuronal function, and endocrine function. Chronic hypogonadism can result in mood instability, impaired learning, impaired memory, weakened bone structure leading to increased risk of fracture, loss of libido which can lead to significant marital or relationship discord, fatigue, loss of muscle tone of the skeletal muscles which results in a reduced metabolic rate, loss of smooth muscle tone leading to intestinal sluggishness and poor absorption of nutrients and minerals, and decreased insulin sensitivity increasing the risk of metabolic syndrome and its associated risks of coronary heart disease. Chronic profound hypogonadism is a modifiable risk factor for breast cancer and osteoporosis in women. With appropriate treatment, risk of breast cancer may be significantly reduced and osteoporosis may be eradicated.

Hypogonadism begins in both women and men at about age 30. It begins with a drop in the levels of androgens which in turn creates a disruption of gamete development which causes a drop in fertility and increasingly unstable and below normal levels of gonadal hormones and higher than normal levels of gonadotropins. As the gonadal hormone levels become more unstable and decline and the gonadotropins continue to rise, the ill health of hypogonadism and the strain on the other endocrine organs begins to appear.

Current treatment of hypogonadism is largely dependent on the age of the patient and personal views of the physician or healthcare provider. Hypogonadism (1-2 gonadal hormones below normal) and profound hypogonadism (3+ gonadal hormones below normal) present with multiple endocrinopathies that, left untreated, cause continuing and worsening health, independent of age. There is no medical basis for not providing treatment as hypogonadism and its progression to profound hypogonadism has the same deleterious effects no matter the patient's age or gender.

There are many benefits of early diagnosis of hypogonadism. With earlier diagnosis treatment can be started sooner resulting in better overall health and general well-being. Earlier treatment would prevent many cases of osteoporosis from ever occurring, significantly reduce risk of breast cancer, and reduce incidence of metabolic imbalances, which in turn would reduce the risk of high blood pressure in turn reducing the risk of heart disease and stroke. Effective early treatment would prevent unnecessary loss of sexual function, sexual response, and genital atrophy.

Currently, diagnosing hypogonadism can only be done with the appropriate lab tests. For men, this is a reasonable method however for women it is not. Diagnosis, like treatment, is largely based on patient age and is often not acknowledged in women of any age. Accordingly, it would be desirable to provide a means for monitoring physiological parameters in women that allow for identifying and treating hypogonadism.

### SUMMARY

Provided herein is a self-contained physiological measuring device adapted for disposition within a patient body cavity, primarily the vagina, for an extended period of time (e.g., 6-48 hours or more). While disposed within the body cavity, the device periodically measures one or more physiological parameters. In addition to measuring such physiological parameters, the device is operative to store such measurements to memory for subsequent download/processing upon removal of the device from the body cavity and/or upon wireless interrogation. Generally the device utilizes passive sensing means to measure one or more parameters while positioned within the body cavity. In this regard, the device is non-invasive in that, while utilized internally, the sensors do not penetrate patient tissue. Therefore, while being utilized internally the device is considered non-invasive.

Generally, the device includes an on-board power supply (e.g., battery), a memory device (e.g., EEPROM or other computer readable media), one or more sensors for taking various measurements and circuitry for controlling the operation of the device. Such circuitry may include firmware, hardware, computer readable memory, software and/or processing capabilities (e.g., a microprocessor or micro-controller). The device is operative to take measurements at predetermine intervals and store such measurements to the memory. Such information may be retrieved from the memory (e.g., upon removal from the body cavity) utilizing either direct interconnection or wireless data transfer. In the latter regard, the device may include a wireless interface (e.g., Bluetooth, RFI, etc.) that allows for transferring data from the memory to an external processing platform (e.g., CPU) for processing and diagnosis purposes. Likewise, the wireless interface may permit programming the device.

The sensors of the device may be any sensors that are deemed appropriate for a particular diagnostic purpose. Such sensors may include, without limitation, strain gauges, pH sensors, pulse oximetry sensors (e.g., LEDs, photo detectors, etc.), temperature sensors, etc. It will be appreciated that strain gauges may be utilized to monitor constriction over time, which may identify, for example, vasoconstriction and vasodilation. A pulse oximetry sensor may determine inter alia, oxygen and/or CO₂ levels. Furthermore, information from one or more of the sensors may be utilized to infer additional physiological parameters including, without limitation, pH, pOH etc.

It will be appreciated that the device may include additional components as well. Such components may include rectifying circuitry that allows for receiving and/or storing energy wirelessly (e.g., from an RF field or a magnetic induction field) while the device is within the body cavity or not. In other embodiments, the device may provide information from the memory while located in the body cavity. That is, the device may include a transmitter that is operative to transmit information wirelessly to an external device.

The components of the device are disposed on a body, which is adapted for insertion into a body cavity. In one arrangement, the body is pliable to allow the device to at least partially deform when inserted through a patient orifice. It may be desirable that the components of the device interconnected to the body are sealed to prevent the intrusion of body fluids. In one arrangement, these components may be encased in a non-permeable material. Such materials may include, without limitation, medical grade silicone. To permit use of a pulse oximetry sensor, it may be desirable that the encasing material be translucent.

In one arrangement, the device is adapted for vaginal insertion. In one such arrangement, the body of the device may be formed in the manner similar to that of a vaginal diaphragm. That is, the body may be formed as a ring. Generally, the body may define an annular ring that is adapted to fit within the vaginal vault. It will be appreciated that when disposed within the vaginal vault the device may be worn for an extended period of time without significantly affecting the activities of the monitored patient. In another arrangement, the body may be generally cylindrical similar in size and shape to, for example, a tampon.

While the self-contained measurement device may be utilized in a number of monitoring situations, the inventor has recognized that such measurements may be particularly apt for diagnosis of gonadal dysfunction and/or failure in female patients. In women, gonadal dysfunction and/or failure is the failure of the ovaries to produce adequate ovarian hormones. During such ovarian dysfunction and/or failure, some or all of the ovarian hormones are below normal level which raises the risk of a number of different illnesses. Such dysfunction and/or failure is also known as hypogonadism. Women suffering from hypogonadism are at risk for osteoporosis, breast cancer, heart disease, periodontal disease and diminished cognitive abilities. Accordingly, it is desirable to monitor patients for decreased ovarian hormone outputs such that hormone replacement therapy can be initiated and/or properly dosed.

The vaginal device allows for monitoring physiological parameters associated with such hormones over an extended period of time. In one arrangement, the vaginal device is worn between 6-48 hours with monitoring taking place every one to five seconds (or other periodic schedule) wherein the device measures oxygen, carbon dioxide, strain/pressure, temperature, and/or other parameters. This information may be subsequently downloaded upon removal of the device for subsequent processing and analysis.

In a related aspect, the device is utilized to generate base line values (e.g., diagnostic markers) that may be applicable to diagnosis of one or more therapeutic conditions, including but not limited to hypogonadism. In this aspect, a plurality of patients may utilize the device over an extended period to obtain one or more parameter measurements of a sample group. Such sample groups may be selected based on, for example, age, ethnicity, and/or the presence or absence of a medical condition. In any arrangement, the sample group of patients utilizes the device internally for a predetermined period of time during which the device takes periodic measurements of one or more physiological parameters. Such parameters may include, without limitation, pulse rate, blood oxygen and/or carbon dioxide levels, strain levels (e.g. constriction), temperature, etc. It will be appreciated that such measurements may be direct measurements or may be inferred or calculated during processing after removal of the device or downloading of information from the device. At the end of the set monitoring period, information from multiple patients is gathered to establish base line characteristics for the sample group. Such base line characteristics may be determined by various known processing techniques. Such known processing techniques may include, for example, regression analysis (or other analysis) to identify the relationship of one or more therapeutic conditions (e.g., hormone levels) to one or more physiological measurements obtained by the device. It will be further appreciated that multiple different physiological measurements may be utilized in conjunction to establish correspondences with one or more therapeutic conditions. For instance, such base line measurements may be a combination of strain and oxygen saturation levels or other values (e.g., pH levels). Such analysis may determine base line values or calibrations for the sample group.

In a further arrangement, first and second or multiple sample groups may be monitored to identify differences between these groups. For instance, a control group may comprise one or more individuals that do not have a particular therapeutic condition (e.g., normal hormone levels). In contrast, one or more test groups may comprise one or more individuals having a particular therapeutic condition (e.g., various elevated or depressed hormone levels). Accordingly, analysis of physiological measurements collected from each of these groups may be gathered and processed to identify differences in the measured values between the groups. Accordingly, such differences in the measured values may subsequently be utilized by, for example, physicians to identify a therapeutic condition and/or the degree of such a therapeutic condition. That is, after such clinical trials, base lines or diagnostic markers may be established for one or more particular therapeutic conditions. Accordingly, a user may wear the device for a predetermined time to non-invasively monitor one or more physiological parameters and these measured parameters may be compared to the established base lines to identify the presence, absence and/or degree of a medical condition. Likewise, therapeutic treatment may be established based on such identification.

In another aspect, the self-contained device may also administer one or more therapeutic agents. To administer such agents, the device includes one or more reservoirs that contain a liquefied or solid therapeutic agent. Such reservoirs may be pressurized such that, upon opening the reservoir, the liquefied or solid therapeutic agent is expelled. For instance, the reservoir may form an elastic bladder that stretches when filled with the therapeutic agent. Alternatively, the device may include an actuator to expel the therapeutic agent from the reservoir. In such an arrangement, the reservoir may include, for example, a plunger that moves in response to an applied signal from the device controller. Other actuators that may be utilized include, without limitation, thin film actuators and micro-pumps.

Typically, when including a reservoir, the device will also include a valve or other means for selectively maintaining the therapeutic agent within the reservoir prior to desired administration. In such an arrangement, the controller may generate a control signal to actuate a valve opening the reservoir or otherwise permitting the therapeutic agent to be displaced from the reservoir. In a further arrangement, the device may include multiple reservoirs. This may allow for providing periodic doses of a therapeutic agent.

### BRIEF DESCRIPTION OF THE FIGURES

FIG. 1 illustrates a perspective view of one embodiment of a self-contained non-invasive internal monitoring device.

FIG. 2 illustrates insertion of the device in FIG. 1 into a body cavity.

FIG. 3 illustrates an exemplary circuit diagram for the device of FIG. 1.

FIG. 4 illustrates a pulse oximeter incorporated onto the device.

FIG. 5 illustrates the inclusion of a reservoir onto the device.

FIG. 6 illustrates a second embodiment of a self-contained non-invasive internal monitoring device.

FIG. 7 illustrates a third embodiment of a self-contained non-invasive internal monitoring device.

FIG. 8 illustrates downloading information from the self-contained monitoring device to an external processing platform.

FIG. 9 illustrates a process for establishing diagnostic markers using the monitoring device.

FIG. 10 illustrates a process for using the monitoring device to measure physiological parameters for comparison to a diagnostic marker(s).

### DETAILED DESCRIPTION

Disclosed herein is a system and method (i.e., utility) for monitoring patient physiological parameters for an extended period of time. The utility utilizes a self-contained measurement/sensing device that is designed for placement within a body cavity. The device includes an onboard power source(s), sensors and an electronic memory for storing physiological measurements taken by one or more of the sensors. As discussed herein, the device is particularly apt for use in intra-vaginal applications and for use in monitoring, diagnosing, and treating hypogonadism. However, it will be appreciated that the utility is not limited to such applications.

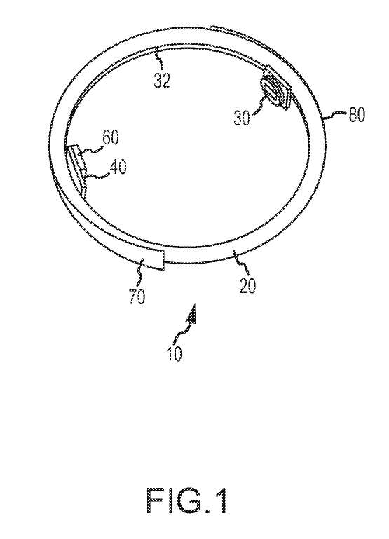

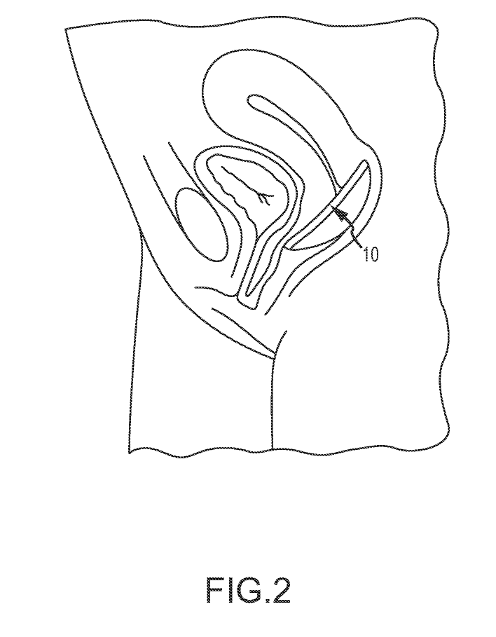

FIG. 1 illustrates one embodiment of a self-contained measurement device that is adapted for vaginal insertion. As shown, the device is formed in a manner that is similar to that of a vaginal diaphragm. In this regard, the body of the device 10 includes an annular ring 20. Generally, the ring 20 is made from a substantially rigid metal or plastic and is coated with a flexible, non-toxic, vaginally acceptable material. Such materials may include silicone rubber or other medical grade silicones. The ring 20 forms a body of the device that supports the measuring/sensory components discussed herein. The ring 20 is shaped and adapted to fit snuggly in the vaginal vault between the posterior aspect of the pubis and the cul-de-sac. See FIG. 2. The annular ring may be generally circular, oval, or other shapes suitably shaped and adapted for placement in the vaginal cul-de-sac, posterior and anterior to the cervix. Generally, the ring will have a diameter between about 50 mm and 80 mm. The size of the ring may be varied to accommodate different individuals.

Referring again to FIG. 1, it is noted that the device 10 includes a plurality of electrical components that provide the sensing and/or measurement function described herein. These components generally include a power source 30, electrical circuitry/control module 40, communications circuitry 50, memory 60 and one or more sensors 70, 80. In general, the electrical components 30-60 are mounted to the inside of the annular ring 20. In various embodiments, one or more sensors 70, 80, etc. are mounted to the outside surface of the annular spring. However, it will be appreciated that all components may be mounted on the inside surface of the ring. The various electrical components 30-60 are interconnected by a flexible circuit board 32. Furthermore, the flexible circuit board 32 may electrically connect one or more sensors 70, 80 to the control module 40.

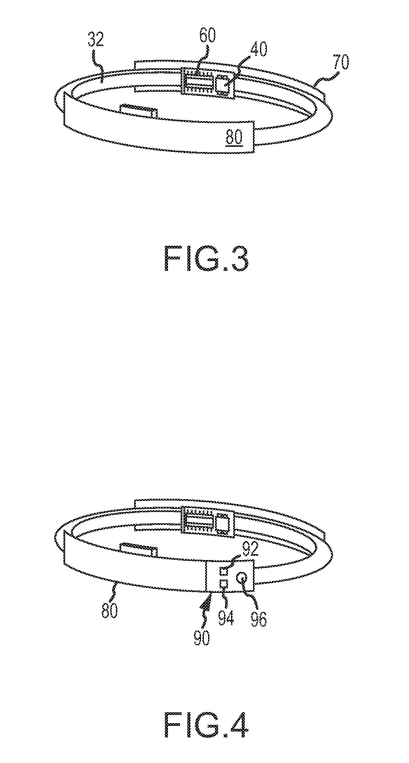

FIG. 3 better illustrates the control module 40 of the device 10. As shown, the control module 40 includes an integrated chip having various firmware defined therein. Furthermore, it would be appreciated that the control module 40 may be programmable to perform functionality required by different measurement sensors variously incorporated into the device. That is, the control module manages the overall device operations. Such device operations typically reside in a computer readable memory as computer instructions (e.g., embedded software). The power source 30 powers the control module 40 and the sensors of the device 10. In the present embodiment, the control module 40 is in operative communication with a wireless transceiver 60, in this case a Bluetooth transceiver. The transceiver is operative to send and/or receive wireless signals for downloading data to a remote computer and/or uploading instructions. Typically, such uploading and downloading will be performed once the device 10 is removed from the body. However, in other embodiments it may be possible and/or desirable to provide wireless transmission while the device is disposed within the body. In one arrangement, the wireless transceiver 60 may further include rectifying circuitry such that power may be provided to the device 10 wirelessly. However, this is not a requirement.

The circuitry includes a memory device at least for storing sensor measurements made by the sensors. The memory may also include operating instructions (e.g., computer instructions) for the device. In one embodiment, EEPROM memory is utilized for the device. The memory device may be programmed with, for example, patient information and/or calibration settings for one or more of the sensors. The type and function of memory incorporated into the device may affect the power requirements of the system. That is, different memories may be utilized based on different requirements and/or intended functions of a given sensor.

In the embodiment of FIG. 3, the device 10 utilizes first and second strain gauges 70 and 80 to allow for measuring constriction within the vaginal wall. Such information may be correlated to determine, for example, vasoconstriction over time. The device also includes a temperature measurement device (not shown). Such a temperature measurement device may be any element that is operable to provide an output signal indicative of temperature. Such a temperature measurement device may include temperature sensitive resisters (i.e. thermisters) and/or thermocouples.

FIG. 4 illustrates another embodiment of a sensor that may be utilized with the device. As shown in FIG. 4, the sensor includes a pulse oximetry sensor 90 on one of the external sensors (e.g., strain gage 80). In this embodiment, the pulse oximetry sensor 90 includes first and second light emitting diodes 92, 94. For instance, the first LED 92 may be a red LED with a wavelength of approximately 660 NM and the other LED 94 may be an infrared sensor having a wavelength between about 900 and 940 NM. It will be appreciated that other wavelengths are possible and within the scope of the present invention. In addition, the sensor 90 includes a photo detector 96 for receiving reflected light. That is, during operation the first and second LEDs are operative to apply light to patient tissue and the photodetector is operative to receive light reflected back from that tissue. In known methods, the ratio of the absorption of the red and infrared light is related to the oxyhemoglobin and deoxyhemoglobin ratio of the patient. That is, processing the information received from the oximetry sensor may provide an estimate of arterial and venous blood oxygen levels. Other physiological information may be obtained or derived from the oximetry information including heart rate and CO₂. Furthermore, use of additional light wavelengths may allow for obtaining measurements of additional characteristics including, without limitation, carbon dioxide. In addition, it will be appreciated that the information from the pulse oximetry sensor may be utilized in conjunction with one or more other measured values and/or calibration values (e.g., in subsequent processing) to infer one or more physiological parameters. Such parameters may include pH, pOH, etc.

It will be appreciated that additional circuitry and/or sensors may be included into the device 10. For instance, the device may include a pH sensor that allows for effectively monitoring the pH of the patient. Embodiments that utilize a direct measuring pH sensor may have sensing components in direct contact with body fluids of the patient. That is, one or more electrodes may extend through the biologically inert coating of medical grade silicone, PTFE, high-density polyethylene (HDPE) or the like.

In embodiments where the electrical components of the device do not come into direct contact with the patient bodily fluids, the device may be reusable. That is, the device may be sterilized and reused on a common patient. Alternatively, in other embodiments the device may allow for sterilization (e.g. autoclaving) such the device may be utilized with different patients. Alternatively, the device may be disposable.

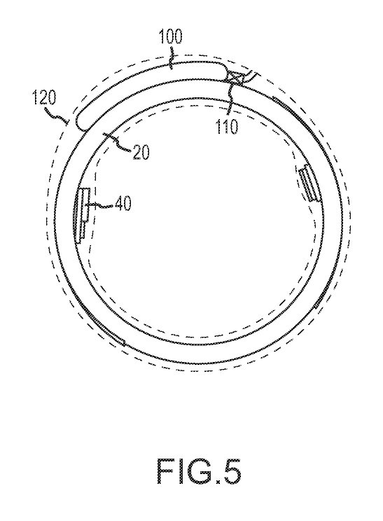

FIG. 5 illustrates another embodiment of the device. In this embodiment, the device is operative to apply and deliver one or more therapeutic agents to a user. The device allows for the controlled delivery of therapeutic agents to the cervix and/or vagina. Such therapeutic agents may include, without limitation, local anesthesia prior to endocervix biopsies, antifungals, anti-infectives, treatments for vaginal and cervical dysplasia and cancer, hormonal therapy etc.

As shown, the device 10 includes a reservoir chamber 100 that contains one or more therapeutic agents. The reservoir chamber is supported on the annular ring 20. It will be appreciated that in use the annular spring and reservoir chamber are also typically encased in a medical grade silicone 120, which may help support the reservoir and/or apply a compressive force to the reservoir. That is, in one embodiment the reservoir chamber 100 is made of a flexible and/or elastic material that may be encased within the silicone prior to the insertion of the therapeutic agent therein. Accordingly, upon insertion of the therapeutic agent, the encasing silicone and/or elastic reservoir apply a compressive force to the contents of the reservoir. This compressive force may assist in displacing the agent from the reservoir chamber when opened.

The reservoir further includes a valve 110 for selectively maintaining the therapeutic agent therein. This valve or a conduit extending there from is typically exposed outside of the encasing silicone to permit the therapeutic agent to be administered to the patient. The valve 110 is operatively connected to the control module 40, which may selectively actuate the valve to permit the controlled release of the therapeutic agent. In operation, one or more therapeutic agents are delivered to and deposited into the reservoir. A manufacturer may apply the therapeutic agent(s) to the reservoir 100 prior to shipping the device, or medical personnel or the patient may apply the therapeutic agent(s) immediately prior to using the device.

In another embodiment, the device may include an actuator (e.g., piezoelectric actuator) for physically displacing fluid from a reservoir. In such an arrangement, the reservoir may operate similarly to a syringe or other compressive force. Likewise, the controller may be operative to control the dosage volume and/or administer multiple doses. In other embodiments, the device includes multiple reservoirs to permit multiple doses and/or the administering of multiple therapeutic agents.

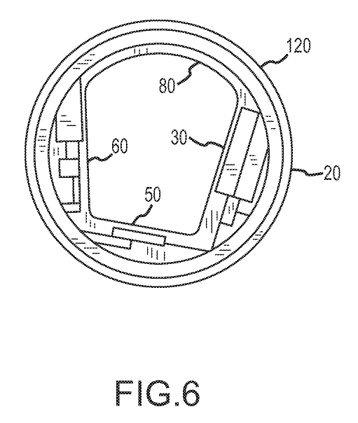

FIG. 6 illustrates another embodiment of the self-contained measurement device 10. As illustrated, this embodiment itself contains a measurement device 10 that again includes an annular ring 20 that forms the body of the device. This ring 20 again supports all the components 30-80 that allow for taking various measurements as discussed above. However, in this embodiment, all of the active components are disposed on the inside surface of the ring 20. As shown, a single strain gauge 80 is disposed about a portion of the inside circumference of the ring 20. Importantly, the strain gauge 80 in this embodiment is spaced from a surface of the ring 20. Specifically, a portion of the elastic bio-inert coating (medical grade silicone, etc.) is disposed between the strain gauge 80 and the surface of the ring 20. In this regard, the strain gauge 80 is operative to move relative to the surface of the relatively rigid ring 20. Accordingly, this embodiment permits the internal strain gauge unfettered movement, which allows the strain gauge to provide more accurate readings.

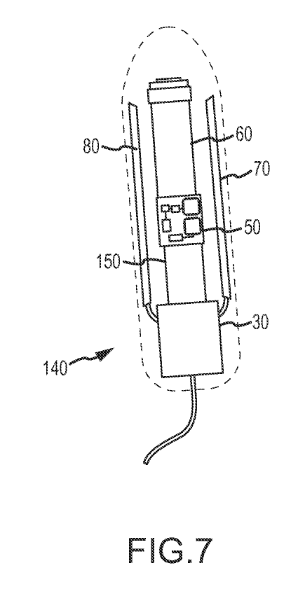

FIG. 7 illustrates another embodiment of the self-contained measurement device. In this embodiment, the device 140 is a generally cylindrical device having a size and shape that is similar to that of a tampon. In this embodiment, the device 140 includes an elongated body onto which the various components 30-60 are mounted. Again, the body 150 is surrounded by bio-inert coating. In this embodiment, one or more strain gauges 80 may extend along the length of the cylindrical device 140. Typically, these strain gauges 80 will be disposed within the bio-inert coating. Of further note, at least a first end of the cylindrical body will include a ring or other attachment point that allows for attaching a string to the device to prevent removal thereof. As with the embodiments discussed above, the device of FIG. 7 may allow for periodic measurements of multiple physiological parameters, the storage thereof and the subsequent download or transfer for processing or other evaluation.

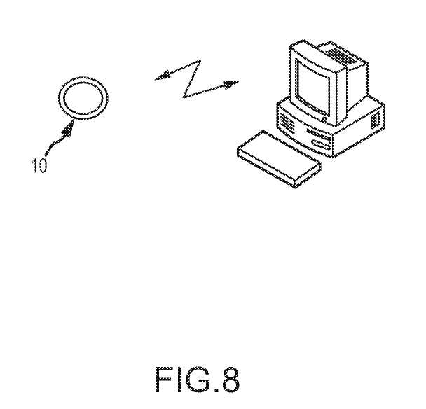

In any of the above-noted embodiments, the measurement device is operative to, upon wireless interrogation or direct interconnection, download accumulated measurements to a processing platform for evaluation. See FIG. 8. The processing platform or computer(s) will include one or more processors or processing units, system memory, and a bus that couples various system components including the system memory to the processor(s). The system memory may include read only memory (ROM) and random access memory (RAM). A basic input/output system (BIOS) containing the basic routines that help to transfer information between elements within computer, such as during start-up, is stored in ROM. These devices may also include internal memory such as a hard disk drive, a magnetic disk drive, and/or an optical disk drive for reading from or writing to a removable optical disk such as a CD ROM, DVD ROM or other optical media.

The drives and their associated computer-readable media provide nonvolatile storage of computer readable instructions, data structures, program modules, and other data for the processing platform. A number of program modules and/or databases may be stored on the hard disk, magnetic disk, optical disk, ROM, or RAM, including an operating system, one or more application programs, other program modules, and programs and data associated with the analysis of the monitored parameters. It will be appreciated that processing and analysis of the monitored data may be variously incorporated into hardware and/or software.

The internal measurement device may be utilized to generate base line values (e.g., diagnostic markers) that may be applicable to diagnosis of one or more therapeutic conditions, including but not limited to hypogonadism. In this aspect, a plurality of patients may utilize the device over an extended period to obtain one or more parameter measurements of a sample group. Such sample groups may be selected based on, for example, age, ethnicity, and/or the presence or absence of a medical condition. In any arrangement, the sample group of patients utilizes the device internally for a predetermined period of time during which the device takes periodic measurements of one or more physiological parameters. Such parameters may include, without limitation, pulse rate, blood oxygen and/or carbon dioxide levels, strain levels (e.g. constriction), temperature, etc. It will be appreciated that such measurements may be direct measurements or may be inferred or calculated during processing after removal of the device or downloading of information from the device.

At the end of the set monitoring period, information from multiple patients is gathered to establish base line characteristics for the sample group. Such base line characteristics may be determined by various known processing techniques. Such known processing techniques may include, for example, regression analysis (or other analysis) to identify the relationship of one or more therapeutic conditions (e.g., hormone levels) to one or more physiological measurements obtained by the device. It will be further appreciated that multiple different physiological measurements may be utilized in conjunction to establish correspondences with one or more therapeutic conditions. For instance, such base line measurements may be a combination of strain and oxygen saturation levels or other values (e.g., pH levels). Such analysis may determine base line values or calibrations for the sample group.

In conjunction with taking measurements from such sample groups, various methods may further include obtaining one or more blood samples such that the measurements from the self-contained monitoring device(s) may be correlated to one or more components found in such blood tests. These components may include, without limitation, hormone levels, and insulin levels It will be further appreciated that members of the sample group may be separated into subgroups based on the level of particular constituent of the blood test. In this regard, the measurement from the devices may be correlated to one or more particular hormones.

For instance, where the device is utilized to monitor hypogonadism, various blood constituents may be measured. Hypogonadism begins in both women and men at about age 30 and typically begins with a drop in the levels of androgens which in turn creates a disruption of gamete development which causes a drop in fertility and increasingly unstable and below normal levels of gonadal hormones and higher than normal levels of gonadotropins. As the gonadal hormone levels become more unstable and decline and the gonadotropins continue to rise, the ill health of hypogonadism and the strain on the other endocrine organs begins to appear.

In this regard, correlating parameter measurements from a sample group having normal levels of gonadotropins may establish a base line reference for one or more passively measurable physiological parameters associated pre-onset of hypogonadism. Likewise, measurements from a sample group having elevated levels of gonadotropins may be analyzed and correlated with one or more passively measurable physiological parameters associated with post-onset of hypogonadism and/or the severity or degree of the condition. In this regard, the self-contained measurement devices discussed above may be utilized to identify diagnostic markers characteristic of hypogonadism or other therapeutic conditions.

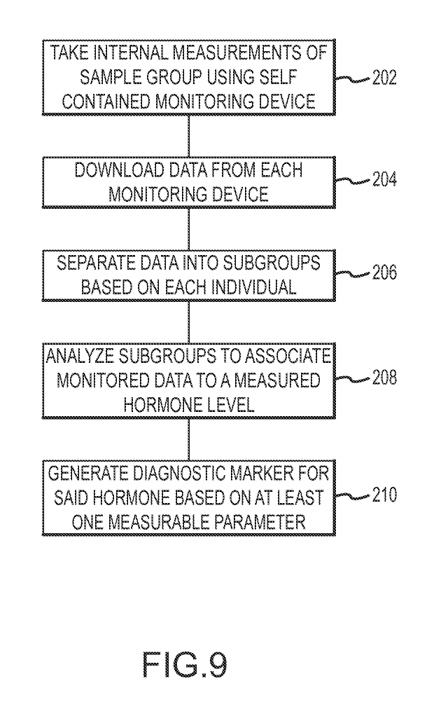

FIG. 9 illustrates a method for establishing a base line reference for a diagnostic marker. Initially, a sample group is monitored 202 for a predetermined period of time using the self-contained measurement device. The monitored parameters from the sample group is then downloaded 204 for processing. The sensor data from different subjects may then be separated 206 into subgroups based on measured levels of a particular blood constituent, hormone, etc. The data for each subgroup may then be analyzed 208 to determine relationships between the measured level and one or more of the monitored parameters for the members of that group. For instance, regression analysis may be performed, which allows for modeling and analyzing several values to determine the relationship between a dependent variable (e.g., a measured hormone level) and one or more independent variables (e.g., oxygen levels, constriction strain levels, etc.). Such analysis may be done for several different subgroups to establish, for instance, relationships associated with differing levels of a particular hormone.

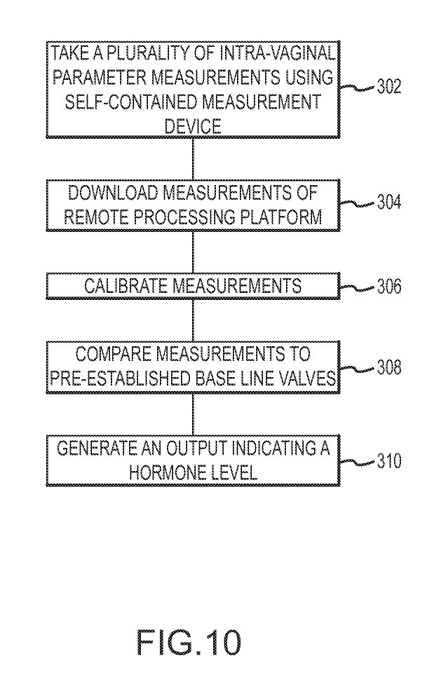

FIG. 10 illustrates a related methodology. As will be appreciated, once a base line level and/or diagnostic marker is established for a sample group, measurements from a single individual may be compared to the base line or diagnostic markers to provide an estimation of the level of one or more hormones and/or the presence or absence of a therapeutic condition. This is illustrated in FIG. 10. Initially, a user inserts the device for a predetermined period to monitor 302 physiological parameters. At the end of the measurement, the data from the measurement device is downloaded 304 to an external computer for processing. Such processing may include calibrating 308 or adjusting the sensor data, reformatting the sensor data, etc. Once downloaded and, if necessary, calibrated/adjusted, data obtained from the individual is compared 308 to pre-established base line values. Such base line values may be stored on a computer readable medium in, for example, a database. Finally, once compared to the base line values, an output may be generated 310 indicating a level of one or more hormones and/or the presence or absence of a therapeutic condition. Accordingly, therapeutic treatment may be prescribed based on the output.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such or other embodiments and with various modifications required by the particular application(s) or use(s) of the present invention. It is intended that the appended claims be construed to include alternative embodiments to the extent permitted by the prior art.

1. A self contained physiological monitoring device adapted for disposition within a body cavity, comprising:
a body adapted for insertion into a body cavity;
a battery mounted to the body;
a memory device mounted to the body;
at least one sensor mounted proximate to the body, the sensor being operatively interconnected to the battery and the memory device, wherein the sensor is operative to monitor at least one physiological parameter and generate an output indicative of the physiological parameter, wherein the output is stored by the memory device; and
a bio-inert coating covering at least a portion of the battery, the memory and the sensor, wherein the bio-inert coating substantially isolates these elements from bodily fluids when disposed within a body cavity.

2. The device of claim 1, wherein the body comprises an annular ring.

3. The device of claim 2, wherein the annular ring comprises a vaginal diaphragm.

4. The device of claim 1, wherein the body is cylindrical.

5. The device of claim 1, further comprising:
control circuitry mounted to the body and being operatively connected to the battery, memory and sensor, wherein the control circuitry controls the operation of the sensor.

6. The device of claim 5, further comprising:
a wireless interface operatively connected to the control circuitry, wherein the wireless interface is at least operative to transmit data from the memory device.

7. The device of claim 4, wherein the control circuit comprises a microprocessor.

8. The device of claim 1, wherein the sensor comprises at least one of:
a temperature sensor;
a pH sensor;
a strain gauge;
a pulse oximetry sensor.

9. The device of claim 1, wherein said sensor comprises a strain gauge, wherein said strain gauge is spaced from the surface of said body.

10. A self-contained intra-vaginal monitoring device, comprising:
an annular body;
a battery mounted to the body;
a memory device mounted to the body;
at least one strain gauge mounted proximate to the body, the strain gauge being operatively interconnected to the battery and the memory device, wherein the strain gauge generates measurement outputs, wherein the measurement outputs are stored by the memory device;
control circuitry mounted to the body and being operatively connected to the battery, memory and strain gauge, wherein the control circuitry controls the operation of the strain gauge and
a bio-inert elastic coating that substantially isolates the body, memory device, control circuit and strain gauge from bodily fluid.

11. The device of claim 10, further comprising:
a pulse oximetry sensor mounted to the body, the pulse oximetry sensor having at least first and second light sources and at least one light detector, wherein the control circuit drives the light sources and the light detector generates outputs, wherein said output is stored by the memory.

12. The device of claim 5, further comprising:
a wireless interface operatively connected to the control circuitry, wherein the wireless interface is at least operative to transmit data from the memory device.

13. A method for generating diagnostic markers associated with hypogonadism in women, comprising:
obtaining, from a plurality of individuals, a plurality of measurements of intra-vaginal physiological parameters, wherein said measurements are taken over an extended monitoring period;
for each individual, associating said parameter measurements with a corresponding level of a measured hormone;
analyzing said parameter measurements and corresponding hormone levels to identify a relationship between at least one physiological parameter and said hormone.

14. The method of claim 13, further comprising;
using different hormone levels of different individuals to generate a base line reference for said measured hormone, wherein said base line reference is based on at least one of said plurality of measurements.

15. The method of claim 13, wherein said method comprises taking intra-vaginal measurements using of at least one of:
a temperature sensor;
a pH sensor;
a strain gauge; and
a pulse oximetry sensor.

16. The method of claim 13, wherein obtaining comprises using self-contained monitoring devices adapted for intra-vaginal insertion to take said measurements.

17. A method for monitoring hypogonadism in a female patient, comprising:
using a self contained measurement device to take a plurality of intra-vaginal parameter measurements over and extended monitoring period;
downloading said parameter measurements from said self-contained measurement device;
processing said parameter measurement to compare said parameter measurements to pre-established baseline values; and
generating an output indicative of a level of hypogonadism.

18. The method of claim 18, further comprising:
based on said output, preparing a treatment schedule.
